(19) United States
(12) Patent Application Publication
(10) Pub. No.: US 2011/0313244 A1
(43) Pub. Date: Dec. 22, 2011

(54) GASTROSCOPE

(76) Inventors: Stefan Förtsch, Kunreuth (DE); Rainer Kuth, Hochstadt (DE); Karl-Heinz Maier, Altdort (DE)

(21) Appl. No.: 13/148,615

(22) PCT Filed: Feb. 15, 2010
(86) PCT No.: PCT/EP10/51853

§ 371 Date: Aug. 9, 2011

(30) Foreign Application Priority Data
Feb. 17, 2009 (DE) 10 2009 009 291.9
May 28, 2009 (DE) 10 2009 023 056.4
Feb. 5, 2010 (DE) 10 2010 006 973.6

(51) Int. Cl. A61B 1/00

(52) U.S. Cl. 600/104

(57) ABSTRACT

A gastroscope has an insertion tube with a working channel therein, and a sensor is guided in the working channel via a guidewire. The sensor has a first electrode produced of an acid-resistant noble metal, and a second electrode produced of silver. An electrical voltage is applied between the first and second electrodes, and a change in an electrical variable is measured between the first and second electrodes when ammonia is present. The gastroscope allows screening of gastric acid and tissue in the stomach lining for Helicobacter pylori in a manner that is gentle for the patient.

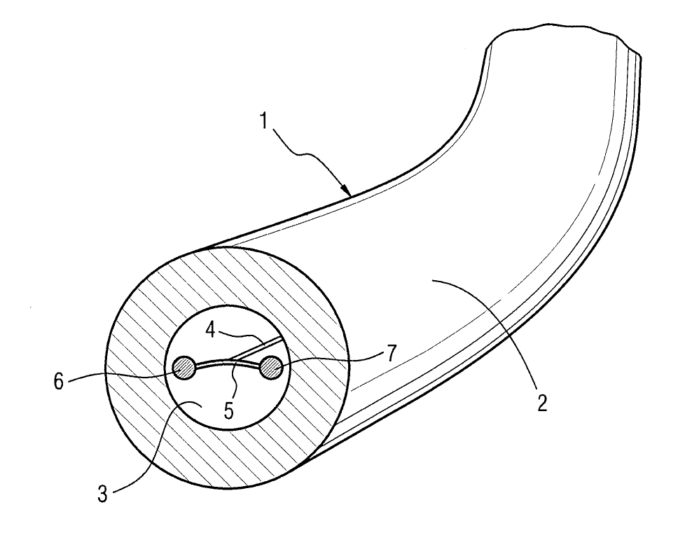

### BACKGROUND OF THE INVENTION

1. Field of the Invention

The invention relates to a gastroscope.

Such a gastroscope, which is a flexible endoscope, serves for examining the upper gastrointestinal tract.

2. Description of the Prior Art

A common cause for discomforts in the upper gastrointestinal tract is a bacterial affliction of the organs therein. For example, an affliction with Helicobacter pylori is held responsible for a vast range of gastric disorders that are associated with an increased secretion of gastric acid. For example, these include type B gastritis, approximately 75% of gastric ulcers and almost all duodenal ulcers. Hence, examining the hollow organs of the gastrointestinal tract for bacteria populations, more particularly Helicobacter pylori populations, is an important component for diagnosing gastric disorders.

For example, Helicobacter pylori is detected using a breath test, in which a patient is administered C-13 masked urea. The C-13 masked CO₂, which is created when urea (CO(NH₂)₂) is split into ammonia (NH₃) and carbon dioxide (CO₂), is detected in the exhaled air. Other methods for detecting Helicobacter pylori are directed at typical blood values such as pepsinogen or gastrin. However, such methods are complex and only have limited reliability. A further test for Helicobacter pylori is the detection of the Helicobacter pylori antigen in fecal matter.

A further option for examining the stomach for a Helicobacter pylori population is provided by so-called gastroscopy. During such an examination, a gastroenterologist takes a tissue sample (biopsy specimen) from the mucosa of the stomach by means of a biopsy in order to examine, either immediately or at a later stage, whether there is an infection with Helicobacter pylori. A known examination method for the tissue sample is, for example, the Helicobacter urease test (HU test, abbreviated HUT). The biopsy specimen is placed into a test medium (measurement solution), which consists of a nutrient solution for this bacteria, urea, and an indicator (litmus). If Helicobacter pylori bacteria is contained in the sample, the bacteria splits the urea (CO(NH₂)₂) using urease into ammonia (NH₃) and carbon dioxide (CO₂). The ammonia then colors the indicator red. The test result is ready after a few minutes. The onset of color change from yellow to red cannot unambiguously be identified in inexpedient conditions.

An alternative to gastroscopy carried out using a flexible endoscope consists of using a so-called endoscopic capsule. Such an endoscopic capsule, which is also referred to as a capsule endoscope or endocapsule, is embodied as a passive endocapsule or a navigable endocapsule. A passive endoscopic capsule moves through the intestines of the patient as a result of peristalsis.

For example, a navigable endocapsule is known from the patent DE 101 42 253 C1 and the corresponding patent application US 2003/0060702 A1, and therein it is referred to as an “Endoroboter” or “endo-robot”. The endo-robot known from these publications can be navigated in a hollow organ (e.g. gastrointestinal tract) of a patient by means of a magnetic field, which is generated by an external (i.e. arranged outside of the patient) magnetic system (coil system). An integrated system for controlling the position, that includes a positional measurement of the endo-robot and automatic regulation of the magnetic field or the coil currents, can be used to detect changes automatically in the position of the endo-robot in the hollow organ of the patient and to compensate for these. Furthermore, the endo-robot can be navigated to desired regions of the hollow organ in a targeted fashion. It is for this reason that this type of capsule endoscopy is also referred to as magnetically guided capsule endoscopy (MGCE).

### SUMMARY OF THE INVENTION

An object of the present invention is to provide a gastroscope that can be used to test the gastric acid and the tissue of the mucosa of the stomach for Helicobacter pylori within a very short period of time.

The gastroscope according to the invention has an insertion tube, in which a work channel is arranged, and a sensor which is guided via a guide wire (e.g. composed of metal or plastic) in the work channel, wherein the sensor comprises a first electrode made of a noble metal, which cannot be attacked by acid (e.g. hydrochloric acid, phosphoric acid, sulfuric acid, gastric acid), and a second electrode made of silver. An electric voltage can be applied between the first electrode and the second electrode, and a change in an electric variable can be measured if ammonia is present between the first electrode and the second electrode.

In the gastroscope according to the invention, ammonia (NH₃) can in a simple fashion be detected directly in the gastrointestinal tract of a patient during the examination, without taking a tissue sample. The gastroscope according to the invention therefore allows an examination of the gastric acid and the tissue of the mucosa of the stomach for Helicobacter pylori that does not put much strain onto the patient.

In a preferred gastroscope according to the invention, the electric voltage between the first electrode and the second electrode equals zero. Thus no current flows between the first electrode and the second electrode. Advantageously, the potential is thereby measured (i.e. without a current) between the first electrode and the second electrode. Thus there hardly is ionic migration in the gastric acid.

In a further advantageous embodiment according to the invention, the electric voltage between the first electrode and the second electrode is an AC voltage with a variably predeterminable frequency spectrum. If gastric acid is exposed to direct current or a directed potential the ions migrate to the associated electrodes, i.e. the cations (e.g. ammonium NH₄⁺) migrate to the cathode and the anions (e.g. chloride Cl⁻) migrate to the anode. By applying a suitable AC voltage, the gastroscope according to the invention reliably prevents complete charging of the first electrode (reference electrode) and complete charging of the second electrode (measurement electrode) because the migration speed of the ions in the gastric acid is almost zero if the frequency is sufficiently high.

When an AC voltage is applied, there is a cyclical change at the second electrode (measurement electrode), which, according to the invention, consists of silver (Ag), between destruction and buildup of the silver chloride (AgCl) layer. Both the destruction of the silver chloride layer and the buildup thereof can be measured by e.g. an impedance measurement and can be compared cyclically. The potential differences and phase differences that can be measured in the process are characteristic for the presence of urease activity, as a result of which presence of Helicobacter pylori can be deduced with a very high certainty.

According to a particularly advantageous embodiment according to the invention, the frequency spectrum of the AC voltage is modulated. As a result, a higher AC voltage stability is obtained, which increases the measurement accuracy and reduces the measurement duration.

In a further embodiment, the electric voltage between the first electrode and the second electrode is a DC voltage, which can be applied for a predeterminable period of time. The predeterminable period of time during which an electric voltage can be applied by the user between the first electrode and the second electrode may lie between zero seconds and continuously, wherein the electric voltage selected by the user may be zero volts or higher. In the case of a period of time of zero seconds or a voltage of zero volts, this is a passive measurement. In the case of values deviating from these, this is an active measurement.

In other embodiments of the gastroscope according to the invention, e.g. potentials, electric currents or electric resistances or the changes therein or variables (e.g. electric conductivity) derived from the electric variables or changes therein can be measured as electric variables.

The second electrode (measurement electrode), which consists of silver (Ag) in the case of the gastroscope according to the invention, must be etched by hydrochloric acid (HCl). This may (but this is not necessary) already occur for the first time before the gastroscope or the second electrode is supplied. However, it is also possible for the users themselves to undertake the initial HCl etching or apply an appropriate silver chloride layer by means of a suitable electrolytic method. After HCl etching or after electrolytic deposition, the second electrode has a silver chloride (AgCl) coating on its surface and is therefore activated for the measurement to detect Helicobacter pylori.

Using the gastroscope according to the invention, ammonia (NH₃) can in a simple fashion be detected directly in the gastrointestinal tract of the patient during the examination, without taking a tissue sample.

The gastroscope according to the invention allows simple open or closed loop control of the sensor or its first electrode (reference electrode) and/or its second electrode (measurement electrode) e.g. by means of a baseline correction. Furthermore, a reproducible regeneration of the sensor, more particularly the second electrode, is possible after each examination.

If the measures outlined above are taken, the second electrode is not completely charged, and so a regeneration of the second electrode only becomes necessary after a multiplicity of examinations.

Moreover, the sensitivity of the sensor and/or its first and/or second electrode can be set in a simple fashion in the gastroscope according to the invention. The sensitivity can be set before and during the examination in respect of Helicobacter pylori.

Platinum (Pt) and gold (Au) can be used as noble metals that are not attacked by acid and therefore are suitable for the first electrode (reference electrode).

After inserting the gastroscope according to the invention, the sensor detects ammonia (NH₃) present in the gastric acid and in the tissue of the mucosa of the stomach on the stomach inner wall. This is used to detect affliction of the tissue (mucosa of the stomach) with Helicobacter pylori in a patient-friendly fashion by detecting ammonia (NH₃). This takes place without a biopsy and therefore puts much less strain on the patient.

The detection of ammonia is a very strong indication for the presence of Helicobacter pylori because ammonia is generated by the Helicobacter pylori bacteria by splitting urea using urease in order to protect itself from the acidic environment of the gastrointestinal tract, more particularly the high acid concentration in the stomach.

As noted above, second electrode (measurement electrode), which consists of silver (Ag) in the gastroscope according to the invention, must be etched by hydrochloric acid (HCl). After the HCl etching, the second electrode has a silver chloride (AgCl) coating on its surface and is therefore activated for the measurement to detect Helicobacter pylori. The activation of the second electrode is based on the following chemical reaction:

Ag+HCl→AgCl+H⁺+e⁻

Since ammonia (NH₃) under normal circumstances does not occur, or only occurs in very low concentrations in a hollow organ of the gastrointestinal tract, such as the stomach, as a result of the following neutralization reaction (forming an ammonium cation by protonation of ammonia)

NH₃+H⁺⇄NH₄⁺

the detection thereof is a very strong indication for the presence of Helicobacter pylori. The proton (H⁺, hydrogen nucleus) is a component of the gastric acid.

The corresponding chemical reaction for detecting Helicobacter pylori is:

AgCl+2NH₃→[Ag(NH₃)₂]⁺+Cl⁻

The AgCl salt (silver chloride) is split into the silver-diammine complex [Ag(NH₃)₂]⁺ and chloride Cl⁻ by ammonia. [Ag(NH₃)₂]⁺ as a cation is very soluble in water and absorbed by the gastric acid. In further embodiments of the gastroscope according to the invention, there is between the first electrode (reference electrode) and second electrode (measurement electrode) either an electric voltage of zero or an electric AC voltage with a variably predeterminable frequency spectrum. Alternatively, a DC voltage can be applied between the first electrode and the second electrode for a predeterminable period of time. In all cases, there is barely any ion migration in the gastric acid (migration speed of the cations and anions is approximately zero).

The electric variable (potential, electric current, electric resistance) measured between the first electrode (reference electrode) and second electrode (measurement electrode) is recorded, displayed, and—if desired—transmitted to evaluation electronics. As a result of an (automated) comparison between the measured value and predetermined values, a possible affliction of the mucosa of the stomach with Helicobacter pylori can be reliably indicated.

After the examination is completed, the electrodes are first disinfected and subsequently rinsed with rinsing solution (hydrochloric acid or a mixture of hydrochloric acid and urea).

By rinsing the second electrode with hydrochloric acid, the silver chloride layer on the second electrode is regenerated. The damage to the silver chloride layer of the second electrode caused by ammonia is thereby removed again. The gastroscope according to the invention can thus once again be used for detecting Helicobacter pylori after a possible necessary recalibration of the sensor. By way of example, the sensor can be calibrated by a dose of synthetic ammonia. After the gastroscopy is completed, the sensor is removed from the work channel and subsequently disinfected. In order to remove still present remains of AgCl it is expedient to clean the sensor using an ammoniacal rinsing solution (e.g. an ammoniacal disinfectant). By a suitable sterilization method a sensor with a suitable design can be reinserted into the gastroscope for another examination.

The gastroscope according to the invention allows an examination, which only puts little strain on the patient, of the mucosa of the stomach in respect of Helicobacter pylori, wherein tissue samples are only taken if the presence of Helicobacter pylori is suspected. The gastroscope can take tissue samples if it has a biopsy apparatus.

### BRIEF DESCRIPTION OF THE DRAWINGS

The single FIGURE schematically illustrates a gastroscope in accordance with the present invention.

### DESCRIPTION OF THE PREFERRED EMBODIMENTS

The invention and further advantageous embodiments are explained in more detail below in the drawing on the basis of a schematically illustrated exemplary embodiment; however, the invention is not restricted to the explained exemplary embodiment.

The only FIGURE shows a gastroscope 1, which is a flexible endoscope and serves for examining the upper gastrointestinal tract.

The gastroscope 1 has an insertion tube 2, in which a work channel 3 is arranged, and a sensor 4 which is guided via a guide wire 5 in the work channel 3. The sensor 4 comprises a first electrode 6 (reference electrode) made of a noble metal, which cannot be attacked by hydrochloric acid, and a second electrode 7 (measurement electrode) made of silver (Ag).

In the illustrated exemplary embodiment both electrodes 6 and 7 have a constant distance from one another.

Platinum (Pt) and gold (Au) can be used as noble metals that are not attacked by hydrochloric acid and therefore are suitable for the first electrode 6.

An electric voltage can be applied between the first electrode 6 and the second electrode 7, as a result of which a change in an electric variable, e.g. potential, electric current, or electric resistance, can be measured if ammonia is present between the first electrode 6 and the second electrode 7.

Further elements such as e.g. optical waveguides (fiber bundles coupled to a light source) and image waveguides (fiber bundles coupled to a camera) and deflection cables (running in the flexible outer shell of the gastroscope) may be arranged in the gastroscope 1 illustrated in the drawing. The further elements, the arrangement of which is known per se, are not illustrated in the drawing for clarity.

Although modifications and changes may be suggested by those skilled in the art, it is the intention of the inventors to embody within the patent warranted heron all changes and modifications as reasonably and properly come within the scope of their contribution to the art.

We claim as our invention:

1-20. (canceled)

21. A gastroscope comprising:
an insertion tube having a longitudinal extent and a work channel proceeding longitudinally inside said insertion tube;
a sensor guided by a guidewire in said work channel;
said sensor comprising a first electrode made of a noble metal, which cannot be attacked by acid and a second electrode made of silver;
a voltage source connected between said first electrode and said second electrode that produces an electric voltage between said first electrode and said second electrode; and
a detector that detects a change in an electrical variable between said first electrode and said second electrode if ammonia is present between said first electrode and said second electrode.

22. A gastroscope as claimed in claim 21 wherein said voltage source produces an electric voltage between said first electrode and said second electrode of zero.

23. A gastroscope as claimed in claim 21 wherein said voltage source produces said electric voltage source as an AC voltage with a variable frequency spectrum.

24. A gastroscope as claimed in claim 21 wherein said voltage source produces said electric voltage as a DC voltage for a predetermined period of time.

25. A gastroscope as claimed in claim 21 wherein said detector measures electrical potential as said electrical variable.

26. A gastroscope as claimed in claim 21 wherein said detector measures electrical current as said electrical variable.

27. A gastroscope as claimed in claim 21 wherein said detector measures electrical resistance as said electrical variable.

28. A gastroscope as claimed in claim 21 wherein said first electrode is made of a noble metal selected from the group consisting of platinum and gold.

29. A gastroscope as claimed in claim 21 wherein said second electrode comprises a silver chloride layer.

30. A gastroscope as claimed in claim 21 wherein at least one of said first electrode and said second electrode is configured to be replaceable.

31. A gastroscope as claimed in claim 21 wherein said second electrode is regenerable.

32. A gastroscope as claimed in claim 21 wherein said insertion tube comprises a work channel, and wherein said sensor is located within the insertion tube next to the work channel.

33. A gastroscope as claimed in claim 21 wherein said sensor is located on an external surface of the insertion tube.

34. A gastroscope as claimed in claim 21 wherein said voltage source generates said electrical voltage as an AC sinusoidal voltage.

35. A gastroscope as claimed in claim 21 wherein said voltage source generates said electrical voltage as an AC triangular voltage.

36. A gastroscope as claimed in claim 21 wherein said voltage source generates said electrical voltage as an AC sawtooth voltage.

37. A gastroscope as claimed in claim 21 wherein said voltage source generates said electrical voltage as an AC voltage representing a noise spectrum.

38. A gastroscope as claimed in claim 21 wherein said voltage source generates said electrical voltage as an AC voltage having a variable frequency spectrum comprised of at least two pulses with respectively different shapes.

39. A gastroscope as claimed in claim 21 wherein said voltage source generates said electrical voltage as an AC voltage with a variable frequency spectrum comprised of components having respectively different bandwidths.

40. A gastroscope as claimed in claim 21 wherein said voltage source generates said electrical voltage as a modulated AC voltage having a variable frequency spectrum.
